(19) United States
(12) Patent Application Publication
(10) Pub. No.: US 2020/0281521 A1
(43) Pub. Date: Sep. 10, 2020

(54) APPARATUS, SYSTEM, AND METHOD FOR MONITORING SLEEP PATTERNS

(71) Applicant: Barbara Cail, Henderson, NV (US)

(72) Inventor: Barbara Cail, Henderson, NV (US)

(21) Appl. No.: 16/295,608

(22) Filed: Mar. 7, 2019

(51) Int. Cl. A61B 5/00; A61B 5/0205

(52) U.S. Cl. CPC A61B 5/4812; A61B 5/746; A61B 5/486; A61B 5/0077; A61B 5/024; A61B 5/742; A61B 5/7475; A61B 5/7405; A61B 5/02055

(57) ABSTRACT

An assembly is disclosed. The assembly has a sleep pattern module, comprising computer-executable code stored in non-volatile memory, a processor, a sensor array, and a device array. The sleep pattern module, the processor, the sensor array, and the device array are configured to sense data of a sleep subject using the sensor array, process the sensed data, issue an alert to a guardian of the sleep subject based on the processed data, control the device array based on the processed data, and provide sleep pattern modification recommendations to the guardian of the sleep subject based on the processed data.

### TECHNICAL FIELD

The present disclosure generally relates to an apparatus, system, and method for monitoring patterns in behavior, and more particularly to an apparatus, system, and method for monitoring sleep patterns.

### BACKGROUND

Existing baby monitors typically collect and transfer audio and/or video data to a device, such as an audio and/or video display, which is monitored by a guardian. Such devices typically provide a real-time stream of an audio recording and/or a video display that a guardian monitors.

Existing baby monitors typically provide the real-time stream of audible sounds and video display of a baby, infant, or child, without recording and further analyzing data associated with the baby. Accordingly, conventional methods do not provide analysis and/or recommendations to the guardian such as analysis and/or recommendations regarding sleep patterns of a baby. Instead, conventional methods merely provide real-time audio recordings and/or a real-time video display to a guardian, with any adjustments to sleep conditions of the baby left entirely to the discretion of the guardian. Accordingly, conventional baby monitors provide no assistance further to providing audio and video information for managing the sleep patterns of a baby.

The exemplary disclosed apparatus, system, and method are directed to overcoming one or more of the shortcomings set forth above and/or other deficiencies in existing technology.

### SUMMARY OF THE DISCLOSURE

In one exemplary aspect, the present disclosure is directed to a system. The system includes a sleep pattern module, comprising computer-executable code stored in non-volatile memory, a processor, a sensor array, and a device array. The sleep pattern module, the processor, the sensor array, and the device array are configured to sense data of a sleep subject using the sensor array, process the sensed data, issue an alert to a guardian of the sleep subject based on the processed data, control the device array based on the processed data, and provide sleep pattern modification recommendations to the guardian of the sleep subject based on the processed data.

In another aspect, the present disclosure is directed to a method. The method includes sensing data of a sleep subject using a sensor array, processing the sensed data, the processed data indicating whether the sleep subject is in a restless state or a deep sleep state, issuing an alert to a guardian of the sleep subject based on the processed data, and controlling a device array based on the processed data, the device array including a soothing device and a lighting device. The method also includes providing sleep pattern modification recommendations to the guardian of the sleep subject based on the processed data, activating the soothing device when the sleep subject is in the restless state, and varying a brightness of the lighting device when the sleep subject is in the deep sleep state.

### BRIEF DESCRIPTION OF THE DRAWINGS

FIG. 1 is a schematic view of an exemplary embodiment of the present invention;

FIG. 2 is a schematic view of an exemplary embodiment of the present invention;

FIG. 3 is a schematic view of an exemplary embodiment of the present invention;

FIG. 4 is a schematic view of an exemplary user interface of the present invention;

FIG. 5 illustrates an exemplary process of an exemplary embodiment of the present invention;

FIG. 6 illustrates an exemplary process of an exemplary embodiment of the present invention;

FIG. 7 is a schematic illustration of an exemplary computing device, in accordance with at least some exemplary embodiments of the present disclosure; and

FIG. 8 is a schematic illustration of an exemplary network, in accordance with at least some exemplary embodiments of the present disclosure.

### DETAILED DESCRIPTION AND INDUSTRIAL APPLICABILITY

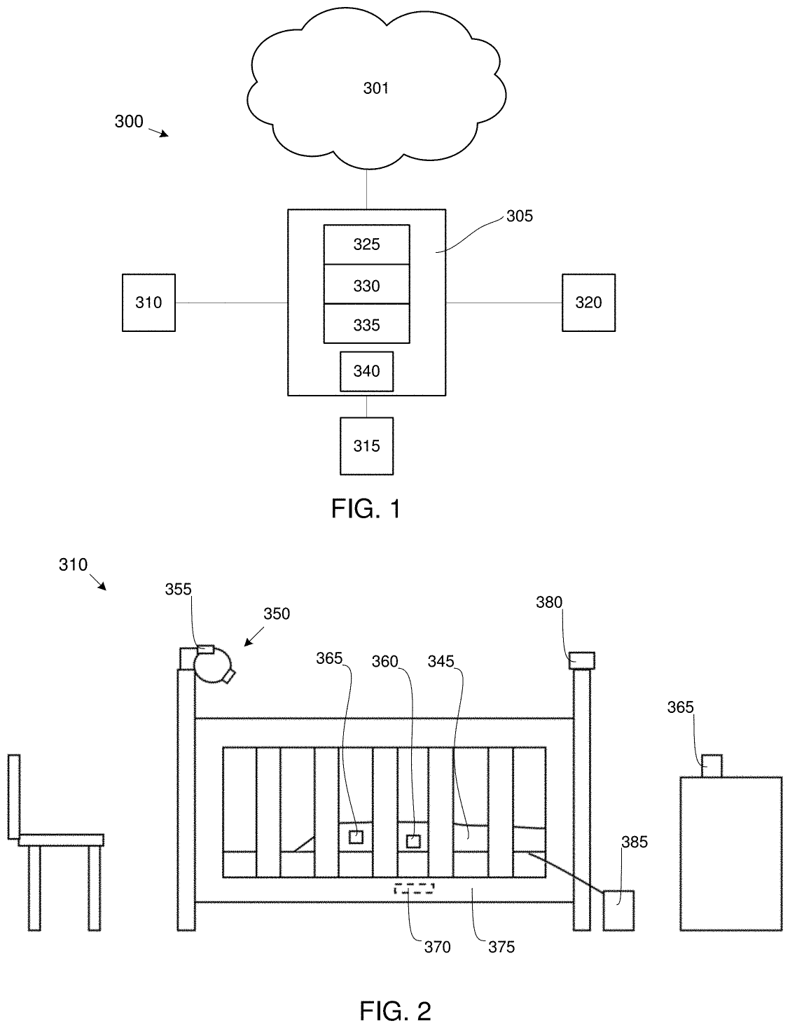

FIG. 1 illustrates an exemplary system 300 for monitoring sleep patterns. For example, system 300 may be used to monitor sleep patterns of an infant or young child. System 300 may be used to monitor sleep patterns of any suitable person of any desired age (e.g., infant, child, adult, or elder person). System 300 may also be used for monitoring sleep patterns of any desired animals such as, for example, mammals or other animals that may be protected in a zoo or other refuge and/or under treatment. System 300 may also be used for monitoring a human in any suitable medical setting such as, for example, a medical diagnostic application, in preparation for, during, and/or following medical treatment, and/or any other suitable application for monitoring sleep as part of medical treatment. System 300 may also be used to provide recommendations for improving sleep quality, duration, and safety of a monitored subject (e.g., infant, young child, or any other suitable subject) to the parent, guardian, and/or caregiver of the subject, with the recommendations for example being individualized to that subject.

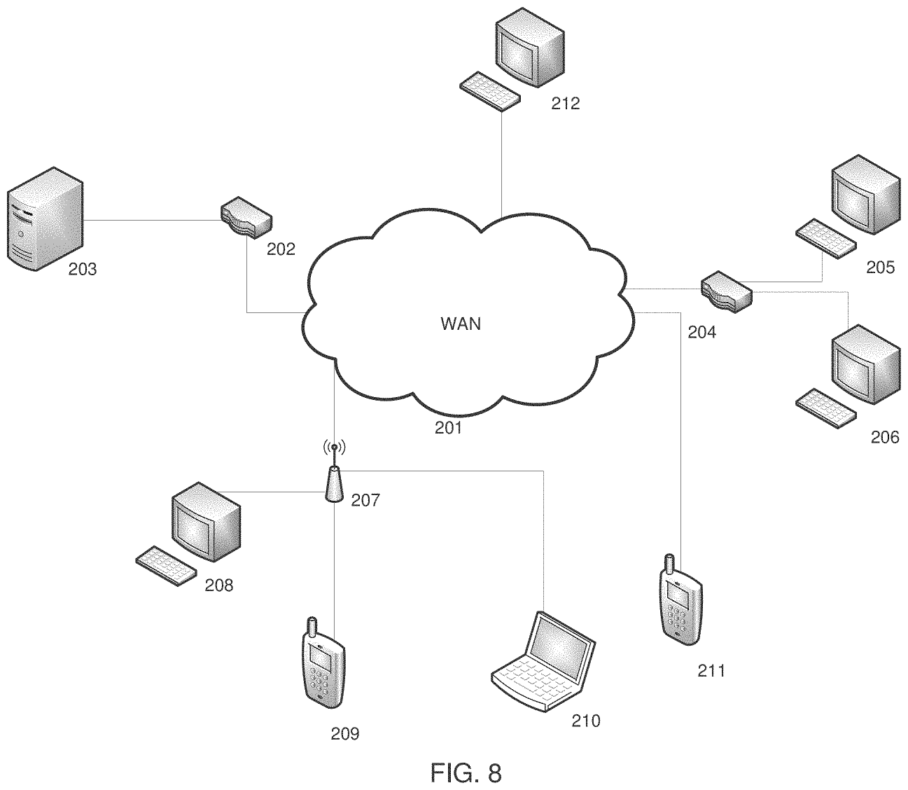

As illustrated in FIG. 1, system 300 may include a subsystem 305, a sensor array 310, a device array 315, and a user interface 320. For example, system 300 may include a plurality of a sensor arrays 310, device arrays 315, and/or user interfaces 320. Subsystem 305, sensor array 310, device array 315, and user interface 320 may be connected for example via network 301, which may be similar to exemplary network 201 disclosed below regarding FIG. 8.

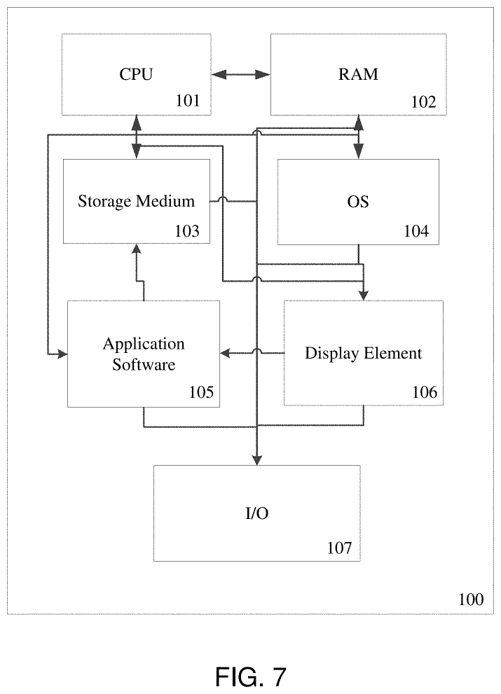

As illustrated in FIG. 1, subsystem 305 may include components similar to the exemplary computing device and network components described below regarding FIGS. 7 and 8. For example, subsystem 305 may include one or more modules having computer-executable code stored in non-volatile memory. Subsystem 305 may also include a processor for processing data associated with system 300 as disclosed herein that may be partially or substantially entirely integrated into any component (e.g., or combination of components) of system 300. Subsystem 305 may for example include a sensor module 325, a device module 330, and an information module 335, which may operate in conjunction with the other components of system 300 as described for example herein. Sensor module 325, device module 330, and information module 335 may each be a separate module or may be integrated into one or more exemplary modules (e.g., one or more sleep pattern modules). Subsystem 305 may include any suitable modules for example for receiving and processing sensed data from sensor array 310 (e.g., and/or device array 315), controlling device array 315 based on the processed data from sensor array 310 (e.g., and/or device array 315), and providing any suitable information to one or more users of system 300.

In at least some exemplary embodiments, sensor module 325 may receive and process any suitable data from sensor array 310 (e.g., and/or device array 315), any other suitable components of system 300, and/or any other suitable source. Sensor module 325 may analyze and/or perform artificial intelligence operations utilizing the sensed data transferred from sensor array 310 (e.g., and/or device array 315) as described for example herein. Device module 330 may control (e.g., provide instructions and/or command data to) device array 315 based on the sensed data from sensor array 310 (e.g., and/or device array 315) processed by sensor module 325 and/or based on input provided by a user of system 300 as described for example herein. Information module 335 may provide any suitable data (e.g., output data such as sleep pattern information) to users of system 300 via user interface 320 as described for example herein.

Subsystem 305 may for example include a controller 340 for controlling an operation of sensor module 325, device module 330, information module 335, sensor array 310, device array 315, user interface 320, and/or any other desired components. Controller 340 may include for example a micro-processing logic control device or board components. Also for example, controller 340 may include input/output arrangements that allow it to be connected (e.g., via wireless and/or electrical connection) to any suitable component of system 300. For example, controller 340 may communicate with components of system 300 via wireless communication, electrical lines, and/or any other suitable communication technique. For example, controller 340 may control sensors of sensor array 310, device array 315, and/or user interface 320 so that components of system 300 act as Internet of Things (IoT) devices that may provide data to and/or be controlled by system 300 as data-providing devices.

Returning to FIG. 1, subsystem 305 may communicate with other components of system 300 via network 301 (e.g., as disclosed below regarding FIG. 8). Subsystem 305 may also be partially or substantially entirely integrated with one or more components of system 300 such as, for example, network 301, user interface 320, sensor array 310, and/or device array 315. Subsystem 305 may include components similar to the exemplary components disclosed below regarding FIGS. 7 and 8. For example, subsystem 305 may include computer-executable code stored in non-volatile memory. Subsystem 305 may also include a processor, or alternatively, a processor for processing data associated with system 300 may be partially or substantially entirely integrated into any portion (e.g., or combination of portions) of system 300.

Subsystem 305 may be configured to retrieve, store, process, and/or analyze data transmitted from one or more sensor arrays 310 (e.g., and/or one or more device arrays 315) to subsystem 305. For example, subsystem 305 may operate using data from any desired number of sensors of sensor array 310 and/or devices of device array 315.

Subsystem 305 may perform analysis using the data received from one or more sensor arrays 310 (e.g., and/or one or more device arrays 315) to for example provide information and recommendations regarding sleep patterns as described herein. For example, subsystem 305 may utilize sophisticated machine learning and/or artificial intelligence techniques to perform predictive analysis using some or substantially all data collected by one or more sensor arrays 310 (e.g., and/or one or more device arrays 315). For example, system 300 (e.g., subsystem 305) may utilize the collected data to prepare and submit (e.g., via network 301, for example via wireless transmission such as via 4G LTE networks) datasets and variables to cloud computing clusters and/or other analytical tools (e.g., predictive analytical tools), which may analyze such data using artificial intelligence neural networks. Subsystem 305 may for example include cloud computing clusters performing predictive analysis. For example, subsystem 305 may utilize neural network-based artificial intelligence to provide information and recommendations for improving sleep quality, duration, and safety based on sensed data (e.g., continuously collected data) transmitted from sensor array 310, device array 315, and/or any other suitable source as described herein. For example, the exemplary neural network may include a plurality of input nodes that may be interconnected and/or networked with a plurality of additional and/or other processing nodes to determine recommendations.

For example, exemplary artificial intelligence processes may include filtering and processing datasets, processing to simplify datasets by statistically eliminating irrelevant, invariant or superfluous variables or creating new variables which are an amalgamation of a set of underlying variables, and/or processing for splitting datasets into train, test and validate datasets using at least a stratified sampling technique. For example, exemplary artificial intelligence processes may also include processing for training a machine learning model to provide recommendations for improving sleep quality, duration, and/or safety based on data collected by sensor array 310 and/or device array 315 as described herein. For example, the prediction algorithms and approach may include regression models, tree-based approaches, logistic regression, Bayesian methods, deep-learning and neural networks both as a stand-alone and on an ensemble basis, and final prediction may be based on the model/structure which delivers the highest degree of accuracy and stability as judged by implementation against the test and validate datasets. Also for example, exemplary artificial intelligence processes may include processing for training a machine learning model to provide recommendations for improving sleep quality, duration, and/or safety based on data collected by sensor array 310 and/or device array 315 as described herein.

For example, system 300 (e.g., subsystem 305) may utilize continuously collected data from sensor array 310 and/or device array 315, which may include thousands, millions, and/or billions of data points, to perform predictive analysis using artificial intelligence and/or machine learning. System 300 (e.g., subsystem 305) may for example use the continuously-growing body of data collected by one or more sensor arrays 310 and/or one or more device arrays 315 to establish benchmarks and metrics for evaluating sleep patterns (e.g., metrics for evaluating sleep quality, sleep duration, and/or sleep safety). For example, system 300 (e.g., subsystem 305) may use substantially all available data to continuously refine predictive analysis for providing recommendations to users to improve sleep quality, duration, and/or safety.

As illustrated in FIG. 2, sensor array 310 may include one or more sensors that may be disposed at or near a subject 345 (e.g., a human such as an infant or young child, an animal, or any other suitable subject).

Sensor array 310 may include a visual sensor 350. Visual sensor 350 may be a camera. For example, visual sensor 350 may be a video camera. Visual sensor 350 may be any suitable video camera such as a digital video camera, a webcam, and/or any other suitable camera for recording visual data (e.g., recording a video or taking pictures). Visual sensor 350 may be for example a three-dimensional video sensor or camera. For example, visual sensor 350 may utilize infrared beams to determine depth data (e.g., using between about 10,000 and about 50,000 infrared beams, for example about 30,000 infrared beams, to determine depth data of corresponding points) to provide three-dimensional imaging of subject 345. For example, visual sensor 350 may utilize infrared beams to track sleep movements of subject 345 by using infrared beams to determine depth data of subject 345 (e.g., acting as a three-dimensional imaging sensor). Visual sensor 350 may include a plurality of cameras or a single camera configured to collect three-dimensional image data. In at least some exemplary embodiments, visual sensor 350 may be a stereoscopic camera and/or any other suitable device for stereo photography, stereo videography, and/or stereoscopic vision. Visual sensor 350 may be an infrared camera or any other suitable thermographic camera for forming a heat zone image. For example, visual sensor 350 may form an image by detecting and recording infrared radiation. Visual sensor 350 may also be a night vision camera or any other suitable camera that may amplify visible light and/or utilize radiation such as electromagnetic radiation.

Sensor array 310 may include an acoustic sensor 355. Acoustic sensor 355 may be a microphone or any other suitable device for detecting and recording noise, vibrations, and/or any other desired noise. Acoustic sensor 355 may include for example one or more cardioid microphones, condenser microphones, omnidirectional microphones, dynamic microphones, and/or any other suitable microphone types. Acoustic sensor 355 may be integrated with and operate in conjunction with visual sensor 350. Acoustic sensor 355 may also be an integrated part of host hardware, such as part of a device similar to user interface 320 (e.g., a mobile phone microphone). Acoustic sensor 355 may also be a separate, stand-alone sensor.

Sensor array 310 may include a biometric sensor 360. Biometric sensor 360 may sense biometric information of subject 345 and/or a guardian (e.g., parent, caretaker, and/or any other suitable guardian) of subject 345. For example, biometric sensor 360 may include one or more wearable devices worn by subject 345 and/or a guardian of subject 345. Biometric sensor 360 may sense any suitable data such as food intake, supplement intake, medicine intake, physical activity and exercise, and/or any other health-related activity. Biometric sensor 360 may sense any desired user attribute such as blood pressure, body temperature, pulse, heartrate (e.g., a heartrate sensor), breathing (e.g., a breathing sensor), movement, weight gain, and/or any other desired characteristic of subject 345 and/or one or more guardians of subject 345.

Sensor array 310 may include one or more sleep sensors 365. Sleep sensor 365 may be integrated with biometric sensor 360 or may be a separate, stand-alone sensor. Sleep sensor 365 may be a wearable device that may be worn by subject 345 or may be disposed under a bedding sheet or on or in a mattress on which subject 345 may be sleeping. Sleep sensor 365 may also be disposed near subject 345 such as, for example, on a table or other location (e.g., in a room in which subject 345 is sleeping). Sleep sensor 365 may be any suitable sensor for sensing sleep data such as a heartrate, breathing, snoring, and/or any other desired sleep characteristics of subject 345. For example, sleep sensor 365 may include an accelerometer and/or any other suitable sensing device for recording movement. Sleep sensor 365 may provide actigraphy data or any other suitable data that may indicate movement during sleep and/or any other suitable data for evaluating sleep patterns of subject 345. Sleep sensor 365 may for example include an echolocation sensor that emits radio waves to measure breathing patterns and/or any other suitable sleep characteristic of subject 345. Sleep sensor 365 may include piezoelectric sensors, motion sensors, radio frequency sensors, and/or any other suitable sensor for measuring sleep patterns.

Sensor array 310 may include a support sensor 370. Support sensor 370 may be disposed on or in a support 375 (e.g., such as a crib, bed, medical treatment table, or any other suitable support that may also include support devices such as mattresses, cushions, pillows, linen, sheets, blankets, and other suitable accessories) that supports subject 345. Support sensor 370 may sense any suitable support data such as a temperature of support 375, moisture content of support 375, pressure exerted by subject 345 on support 375, and/or any other desired attribute of support 375. Support sensor 370 may include components that may be similar to at least some of the various exemplary sensors described herein.

Sensor array 310 may include an ambient condition sensor 380. Ambient condition sensor 380 may include a barometer or any other suitable device for sensing a barometric pressure (e.g., ambient pressure) of a space (e.g., room) in which subject 345 is located. Ambient condition sensor 380 may sense any suitable ambient condition data of a space in which subject 345 is located. Ambient condition sensor 380 may include any suitable sensing components for sensing temperature (e.g., an ambient temperature sensor) such as, for example, a thermocouple, a resistance temperature detector (RTD), a thermistor, and/or a semiconductor-based temperature sensing component. Ambient condition sensor 380 may also include a hygrometer or any other suitable device for sensing a relative humidity of a space in which subject 345 is located. Ambient condition sensor 380 may also sense air quality (e.g., presence and/or amount of various particles of material present in air surrounding subject 345) and any other desired data regarding conditions of a space (e.g., room) in which subject 345 is disposed.

Sensor array 310 may also include a medical sensor 385 (e.g., and/or sensor module 325 may receive data from any suitable medical, diagnostic, and/or other treatment equipment such as hospital equipment and other medical equipment). For example, medical sensor 385 may be a medical sensor that senses a biometric attribute of subject 345. For example, medical sensor 385 may sense any suitable medical data associated with devices (e.g., data based on an operation of these exemplary devices) such as a cardiac monitor, an invasive blood pressure monitor, a ventilator, a temperature control device, a dialysis machine, an ECMO machine, and/or any other suitable medical device for treating and/or monitoring subject 345.

The exemplary sensors of sensor array 310 described above may be integrated into one or more sensor arrays located in any suitable location relative to subject 345. Sensor module 325 may communicate with, control, and/or exchange data with the exemplary sensors of sensor array 310 (e.g., as well as any other suitable device or data source that may provide data for processing to sensor module 325). For example in addition to data received from sensor array 310, sensor module 325 may receive data from external databases (e.g., hospital records or other medical records, data from the literature regarding sleep hygiene and/or sleep patterns, aggregate data compiled from sleep studies and other suitable research, and/or any other suitable sleep pattern information), direct input from users of system 300, and/or any other suitable data source (e.g., received via network 301).

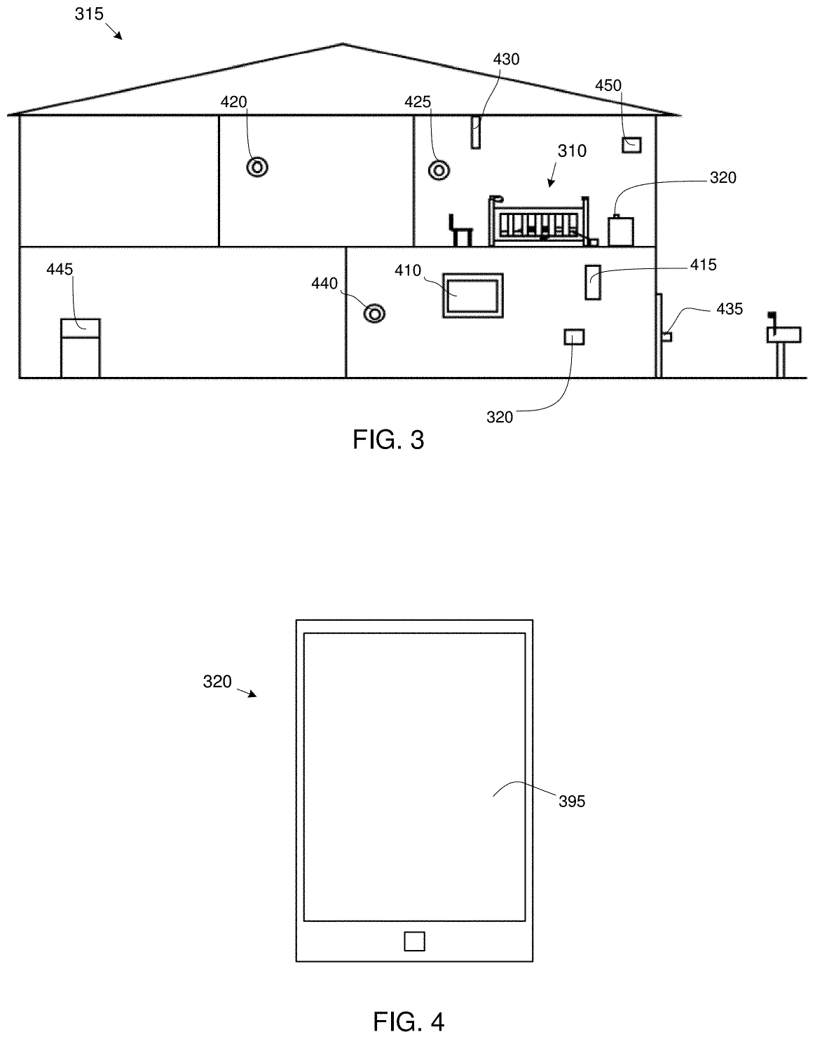

As illustrated in FIG. 3, device array 315 may include one or more devices that may be co-located with subject 345 (e.g., in the same room, house, or building as subject 345 or in the same area of a facility such as a medical facility as subject 345). Device array 315 may for example include a plurality of household and/or commercial devices that may be networked together (e.g., and with other components of system 300) as an Internet of Things network of devices that interact and exchange data.

Device array 315 may include a plurality of entertainment devices 410 and 415. Entertainment devices 410 and 415 may be for example televisions, smartboards, video game systems, speaker or sound systems (e.g., a home sound system), radios, stereos, and/or any other desired residential or commercial equipment.

Device array 315 may include a plurality of utility devices 420, 425, 430, 435, 440, 445, and 450. Utility devices 420, 425, 430, 435, 440, 445, and 450 may be for example thermostats, lighting devices (e.g., lighting components) such as lamps and overhead lighting, doorbells, security systems, alarms such as security alarms and fire alarms, and/or kitchen or food preparation devices (e.g., such as refrigerators, ovens, microwaves, toasters, and coffee makers that may for example have alarms). For example, utility device 430 may be a lighting element disposed in a room in which subject 345 may be sleeping. Also for example, utility device 425 may be a thermostat that controls a temperature (e.g., and/or other ambient conditions) within the room in which subject 345 may be sleeping. Further for example, utility device 450 may be a soothing device such as a lighting display (e.g., a lighting display device that may emit soothing light), a soothing vibration device (e.g., a device that may vibrate to provide soothing to a baby or child), and/or a mobile activation device (e.g., a device that may provide soothing movement to a baby or child, or move an object within a field of view of a baby or child that may soothe the baby or child). For example, utility device 450 may be a soothing device that is an audio device (e.g., sound soother or similar device that may provide comforting sounds to subject 345 for example during sleep).

One or more user interfaces 320 may also be co-located in the exemplary space illustrated in FIG. 3. For example, user interfaces 320 that may be smart speaker devices (e.g., Alexa, Siri, HomeKit, and/or any other suitable smart speaker techniques) may be located in one or more rooms of an exemplary space (e.g., in the same and/or different rooms than a room in which subject 345 is located), as well as other exemplary user interfaces 320 described herein (e.g., smartphones, tablets, and/or computers).

User interface 320 may be any suitable user interface for receiving input and/or providing output (e.g., raw data and/or results of predictive analysis described above such as recommendations) to a user. For example, user interface 320 may be a touchscreen device (e.g., of a smartphone, a tablet, a smartboard, and/or any suitable computer device), a computer keyboard and monitor (e.g., desktop or laptop), an audio-based device for entering input and/or receiving output via sound, a tactile-based device for entering input and receiving output based on touch or feel, a dedicated user interface designed to work specifically with other components of system 300, and/or any other suitable user interface (e.g., including components and/or configured to work with components described below regarding FIGS. 7 and 8).

In at least some exemplary embodiments, user interface 320 may include haptic feedback components or haptics for providing information to users of system 300. For example, user interface 320 may be configured to vibrate and/or exert predetermined pressure against a user's hand or other body part to represent a movement or condition occurring with a subject (e.g., infant or young child) being sensed by sensor array 310. For example, user interface 320 may be a smartphone or tablet configured to vibrate and/or may include components configured to transfer haptic data to a user. User interface 320 may also include virtual reality components (e.g., virtual reality glasses, gloves, and/or other suitable equipment) that may provide a user with a virtual representation of an area in which a subject (e.g., infant, young child, or other suitable subject) is located. User interface 320 may also include augmented reality (AR) components such as visual overlays that may be displayed over image data recorded by visual sensor 350. For example, user interface 320 may include AR visualization such as labeling hazards, providing visual warnings, providing statistics of subject 345 as overlays, and/or any other desired AR display elements. For example, a virtual representation of a room in which the subject is located may be produced based on data sensed and processed by system 300. User interface 320 may also include haptic feedback equipment that may operate in conjunction with the virtual reality equipment that user interface 320 may include. For example based on an operation of virtual reality and haptic feedback equipment of user interface 320, a user may virtually move close to and/or feel a chest area or other portion of the subject (e.g., to check breathing and/or heartbeat) based on data provided by sensor array 310 and processed by sensor module 325.

For example, user interface 320 may include a touchscreen device of a smartphone or handheld tablet. For example as illustrated in FIG. 4, user interface 320 may include a display 395 (e.g., a computing device display, a touchscreen display, and/or any other suitable type of display) that may provide raw data and/or predictive analysis results to a user. For example, display 395 may include a graphical user interface to facilitate entry of input by a user and/or receiving output. For example, a user may utilize user interface 320 to query raw data results and/or enter parameters to define a set of desired output (e.g., areas identified by the user as including issues such as sleep pattern challenges to be resolved). Also for example, system 300 may provide alerts to a user (e.g., based on an operation of information module 335) via output transmitted to user interface 320 (e.g., alerts pushed to a user via user interface 320) as described herein. System 300 may also send alerts to users by alternative methods such as, for example, via text message, email, and/or recording sent by telephone.

In at least some exemplary embodiments, user interface 320 may include applications (e.g., that may be accessed and operated using a smartphone and/or tablet) and/or websites (e.g., that may be accessed and operated using a laptop or desktop computer) that a user may utilize to provide input to and receive output from system 300. In at least some exemplary embodiments, user interface 320 may be or may include smart speaker components (e.g., Alexa, Siri, HomeKit, and/or any other suitable smart speaker techniques).

In at least some exemplary embodiments, user interface 320 may include any suitable component for conducting a conversation with a user via textual, auditory, or other suitable techniques. For example, user interface 320 may include a chatbot such as an artificial intelligence chatbot. The exemplary chatbot may communicate with a user of system 300 such as a guardian of a subject (e.g., infant, child, adult, elder person or any other suitable subject) to request feedback from the guardian, provide suggestions or recommendations to the guardian (e.g., based on processing by system 300 as described herein), ask for further details regarding waking behavior or other input, and/or request or provide any other desired information. The exemplary chatbot may for example facilitate the data exchange in a conversational manner (e.g., via auditory conversation or textual chat).

The exemplary disclosed apparatus, system, and method may be used in any suitable application for monitoring sleep patterns. For example, the exemplary disclosed apparatus, system, and method may be used in any application for monitoring sleep patterns of an infant or young child. The exemplary disclosed apparatus, system, and method may be used to monitor sleep patterns of any suitable person of any desired age (e.g., infant, child, adult, or elder person). The exemplary disclosed apparatus, system, and method may be used to monitor sleep patterns of any desired animals such as, for example, mammals or other animals that may be protected in a zoo or other refuge and/or under treatment. The exemplary disclosed apparatus, system, and method may also be used for monitoring a human in any suitable medical setting such as, for example, a medical diagnostic application, in preparation for, during, and/or following medical treatment, and/or any other suitable application for monitoring sleep as part of medical treatment. The exemplary disclosed apparatus, system, and method may for example be used in a residence, a medical or psychiatric facility (e.g., an inpatient facility such as a hospital), and/or any other suitable facility or location for monitoring and/or analyzing sleep patterns or behavior. The exemplary disclosed apparatus, system, and method may also be used in any suitable application involving providing recommendations for improving sleep quality, duration, and safety of a monitored subject (e.g., infant, young child, or any other suitable subject) to a parent, guardian, and/or caregiver of that subject.

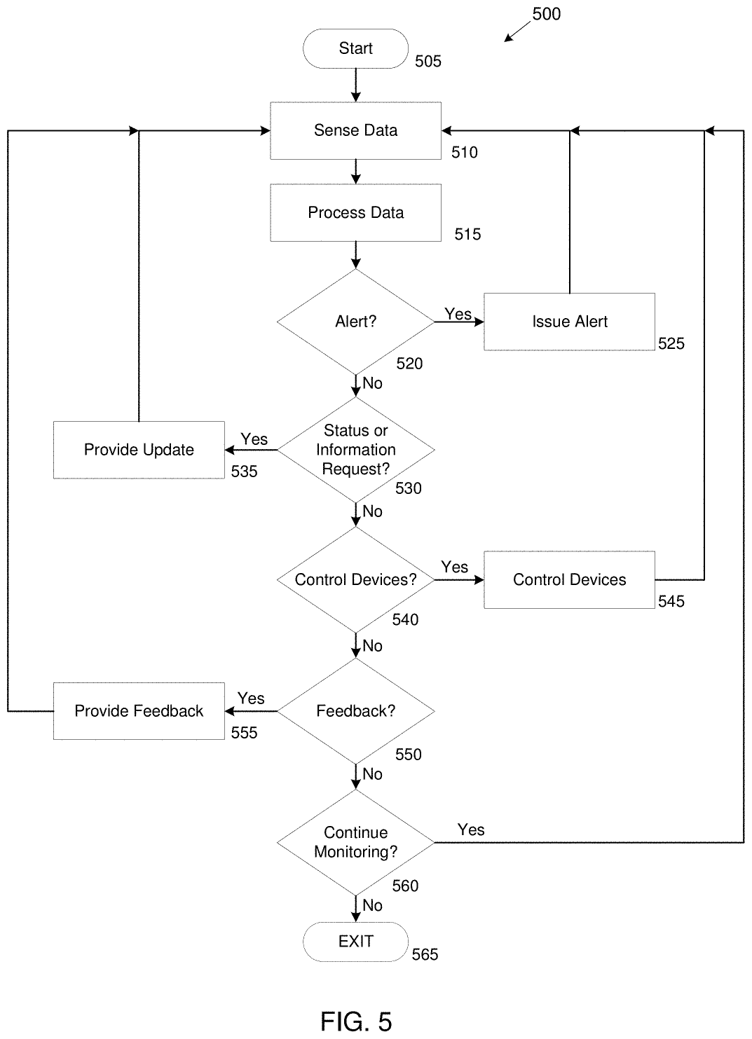

An exemplary operation of the exemplary disclosed apparatus, system, and method will now be described. For example, FIG. 5 illustrates an exemplary process 500. Process 500 starts at step 505. At step 510, the exemplary sensors of sensor array 310 may sense data associated with subject 345 and/or conditions of the space in which subject 345 is located, and this data may be transferred to sensor module 325. For example, one or more visual sensors 350 may sense any suitable visual data for example as described above and transfer the data to sensor module 325. One or more acoustic sensors 355 may sense any suitable acoustic data for example as described above and transfer the data to sensor module 325. One or more biometric sensors 360 may sense any suitable biometric data for example as described above and transfer the data to sensor module 325. One or more sleep sensors 365 may sense any suitable sleep data for example as described above and transfer the data to sensor module 325. One or more support sensors 370 may sense any suitable support data for example as described above and transfer the data to sensor module 325. One or more ambient condition sensors 380 may sense any suitable ambient condition data for example as described above and transfer the data to sensor module 325. One or more medical sensors 385 may sense any suitable medical data for example as described above and transfer the data to sensor module 325. Data from external databases, direct input from users of system 300, and/or any other suitable data source as described for example above may be transferred to sensor module 325. The exemplary data may be transferred from sensor array 310 and/or any other suitable exemplary source to subsystem 305 (e.g., sensor module 325) either continuously or at any desired constant or variable intervals in real-time or near real-time.

At step 515, subsystem 305 (e.g., sensor module 325 and/or information module 335) may process the data collected and transferred at step 510 using any suitable data processing and/or artificial intelligence techniques. For example, subsystem 305 (e.g., sensor module 325 and/or information module 335) may analyze biometric data (e.g., and/or visual data, acoustic data, sleep data, support data, ambient condition data, or medical data) collected from observation (e.g., sensing by sensor array 310) of subject 345 (e.g., a sleeping infant, sleeping child, or other suitable subject). Subsystem 305 (e.g., sensor module 325 and/or information module 335) may use machine learning and deep learning to analyze streaming biometric input data (e.g., and/or visual data, acoustic data, sleep data, support data, ambient condition data, or medical data) transferred from sensor array 310 or other suitable exemplary sources described for example herein. Exemplary computing devices and/or neural networks of system 300 may operate using any suitable technique for data processing and/or machine learning as described for example herein. System 300 may issue alerts, control exemplary devices of device array 315 and sensor array 310, and provide feedback and recommendations to users based on the exemplary data processing techniques and artificial intelligence operations described for example herein.

At step 520, system 300 (e.g., subsystem 305) may determine whether to issue an alert based on the data sensed at step 510 and processed and analyzed at step 515. System 300 may proceed to step 525 as described below if issuing an alert is appropriate based on the data sensed and analyzed at steps 510 and 515. If issuing an alert is not appropriate, system 300 may proceed to step 530 as described below.

At step 525, system 300 (e.g., subsystem 305) may issue an alert to users of system 300 and/or other parties such as first responders. For example, information module 335 may cause an alert to be issued based on the data sensed at step 510 and processed and analyzed at step 515. In at least some exemplary embodiments, subsystem 305 may communicate with and send notifications and alarms to other devices such as user interface 320. For example, subsystem 305 may cause user interface 320 (e.g., a smartphone, a wearable device, and/or a smart speaker such as Alexa and other exemplary devices described above) to issue any suitable alarm (e.g., audible alarm such as a beeping or siren, visual alarm such as a flashing light, vibrational alarm such as causing user interface 320 to vibrate, and/or any other suitable alert). Subsystem 305 may issue the exemplary alert or alarm to notify a guardian of subject 345 via an alarm emitted by user interface 320 and/or device array 315 (e.g., and/or sensor array 310) based on the data sensed at step 510 and processed and analyzed at step 515 (e.g., based on artificial intelligence operation of information module 335). In at least some exemplary embodiments, subsystem 305 may activate an emergency medical system that may for example issue an alert to security personnel and/or authorities (e.g., police, medical personnel, and/or other first responders) based on predetermined criteria and/or commands or settings provided by a user of system 300. For example, subsystem 305 may place a call directly using 9-1-1 to report certain circumstances based on predetermined criteria or machine learning. For example, subsystem 305 may activate an emergency medical system (e.g., activate EMS) when sensor array 310 includes medical sensor 385 that may indicate a life-threatening situation to subject 345 and/or when sensor array 310 provides data (e.g., or lack of data) that may indicate a threatening situation to subject 345.

At step 525 in at least some exemplary embodiments, system 300 (e.g., subsystem 305) may issue an alert to a guardian of subject 345 when sensed data of subject 345 is not being received from some or all of biometric sensor 360, sleep sensor 365, and/or medical sensor 385. The alert may be issued when an absence of data or analysis of received data indicates that subject 345 may be experiencing a stoppage or interruption in breathing and/or other indicators of a SIDS (sudden infant death syndrome) event. System 300 may issue such a SIDS alert using some or all devices of user interface 320, sensor array 310, and device array 315 (e.g., visual and audio alerts emitted from entertainment devices, smartphones, flashing housing lights, audible smart speaker alarms, and/or any other suitable warning that may alert guardians of subject 345). Alerts may be accompanied by recommended actions to take (e.g., actions such as resuscitation and other medical or treatment actions).

At step 525 in at least some exemplary embodiments, system 300 (e.g., subsystem 305) may issue a notification to a guardian of subject 345 when data sensed at step 510 and processed and analyzed at step 515 indicates that subject 345 is awake or restless. System 300 may issue notifications indicating whether subject 345 is in a deep sleep state (e.g., deep state of sleep), REM sleep, light sleep, transitioning between sleep phases (e.g., a transitional sleep state), restless state of sleep, in a waking state, or awake. System 300 may issue notifications indicating whether subject 345 is following normal sleep patterns or deviating from normal sleep patterns. System 300 may issue any desired notification based on data sensed at step 510 and processed and analyzed at step 515. After issuing an alert or notification at step 525, system 300 may return to step 510.

At step 530, system 300 (e.g., subsystem 305) may determine whether a status or information request has been received from a user or whether a user should be prompted for information based on the data sensed at step 510 and processed and analyzed at step 515. System 300 may proceed to step 535 as described below if a status or information request is appropriate. If a status or information request is not appropriate, system 300 may proceed to step 540 as described below.

At step 535, system 300 (e.g., subsystem 305) may receive an information request from a user of system 300 such as a guardian of subject 345. The requesting user may input the request via any suitable technique such as by using user interface 320 (e.g., typing or making a voice request via a smartphone application, computing device, smart speaker such as Alexa, or any other suitable exemplary user interface described herein). The requesting user may request and receive a status (e.g., sleep status) of subject 345 from system 300. The requesting user may request that system 300 may serve as a baby monitor and provide video and/or audio using sensor array 310. The requesting user may direct system 300 to turn and/or zoom sensors of sensor array 310 to provide desired resolution and information scope (e.g., zooming or turning visual sensor 350 and/or controlling other sensors of sensor array 310). The requesting user may request and receive a real-time visual, audio, and/or haptic representation (e.g., breathing or heartbeat) and/or virtual reality representation of data sensed by sensor array 310 as described for example above. System 300 may provide continuous or periodic status updates of subject 345 based on predetermined criteria and/or data sensed at step 510 and processed and analyzed at step 515.

Also at step 535, system 300 may prompt a user of system 300 such as a guardian of subject 345 for information based on data sensed at step 510 and processed and analyzed at step 515. System 300 may prompt the user for information using any suitable technique such as via user interface 320 (e.g., textually or audibly). System 300 may for example request feedback from a user or request details of a behavior of subject 345 such as a waking or restless behavior. In at least some exemplary embodiments, system 300 may also provide feedback to a guardian of subject 345 as to whether or not subject 345 is present based on data sensed by sensor array 310 at step 510. For example, system 300 may prompt a guardian of subject 345 to answer a question such as “Did you just lay your child down for his/her nap?” based on sensing that subject 345 is not present under conditions in which subject 345 is expected to be present (e.g., during a nap). For example when subject 345 is not present during an unexpected time (e.g., not present in a crib during nap time), system 300 may prompt the guardian to check on the child and/or issue an alert to the guardian.

System 300 may utilize a chatbot as described for example above during data exchange with a user at step 535. System 300 may audibly and/or visually request feedback from a user, provide suggestions to the user, and/or ask for details about subject 345 or an area in which subject 345 is located, e.g., in a conversational manner. After responding to an information request or requesting and receiving information from a user at step 535, system 300 may return to step 510.

At step 540, system 300 (e.g., subsystem 305) may determine whether one or more devices of device array 315 (and/or sensor array 310) are to be controlled based on the data sensed at step 510 and processed and analyzed at step 515. System 300 may proceed to step 545 as described below if controlling an exemplary device is appropriate. If controlling an exemplary device is not appropriate, system 300 may proceed to step 550 as described below.

At step 545, system 300 (e.g., subsystem 305) may control one or more devices of device array 315 (e.g., and/or sensor array 310) based on the data sensed at step 510 and processed and analyzed at step 515. For example if subject 345 is in a waking, restless, or awake state based on analysis of the sensed data, device module 330 may control device array 315 including, e.g., entertainment devices such as televisions and speakers having adjustable volume, appliances such as ovens and microwaves having adjustable timers, and/or utility devices such as doorbells to operate in a low-volume or muted mode. For example, when subject 345 is awake or at a sleep stage where waking is likely based on analysis of the sensed data (e.g., a restless state), device module 330 may instruct device array 315 (e.g., various devices in a home or other location) to operate in a quiet or silent mode until subject 345 enters a relatively deeper sleep. In addition to controlling devices to operate with lower volume, device module 330 may control lighting elements of device array 315 to be lowered or dimmed, control a temperature of thermostats to be adjusted, and/or control any other devices of device array 315 to operate in a manner that facilitates increasing probability of lulling subject 345 into a relatively deeper sleep state. Device module 330 may also control speakers, soothing devices (e.g., lighting display, vibration device, mobile activation device, and/or sound soothers as described for example above), and/or other audio devices of device array 315 to play soothing music when subject 345 is waking, awake, or restless. Device module 330 may also control devices of device array 315 (e.g., and/or sensor array 310) to operate normally or relatively loudly when subject 345 is in a relatively deep sleep state or when desired by a user of system 300 such as a guardian of subject 345. Device module 330 may control one or more devices of device array 315 (e.g., and/or sensor array 310) to operate in any desired manner based on data sensed at step 510 and processed and analyzed at step 515 (e.g., based on an operation of information module 335). After control of one or more devices at step 545, system 300 may return to step 510.

At step 550, system 300 (e.g., subsystem 305) may determine whether providing feedback to a user of system 300 is appropriate based on the data sensed at step 510 and processed and analyzed at step 515. System 300 may proceed to step 555 as described below if providing feedback is appropriate. If providing feedback is not appropriate, system 300 may proceed to step 560 as described below.

At step 555, system 300 (e.g., subsystem 305 including information module 335) may provide any desired information and feedback to a user of system 300 based on the exemplary artificial intelligence and machine learning operations described for example herein. Information module 335 may for example provide sleep training information that may assist a user such as a parent or guardian of subject 345 to improve sleep patterns, sleep amounts, and quality of sleep of subject 345. Information module 335 may perform data analysis (e.g., biometric data analysis and/or analysis of any of the sensed data described herein) using artificial intelligence (e.g., machine learning or deep learning) to make behavior modification recommendations to provide suggested actions to take relating to subject 345. Information module 335 may use data of current sleep science and pediatric standards in analysis to generate the recommendations. Information module 335 may also provide recommendations regarding specific concerns, based on guardian feedback. For example, information module 335 may track sleep patterns against specific behavioral issues and/or heath concerns (e.g., ADHD, childhood anxiety or depression, autism spectrum disorders, and/or any other issue or concern). The sleep patterns may be tracked against behavior changes to provide insights and/or recommendations. Information module 335 may also be able to provide insights against performance data (e.g., academic, physical, social, and/or other desired performance data) collected from a guardian of subject 345. Information module 335 may recommend actions to take that are age-appropriate (e.g., newborn, infants, toddlers, and any other desired stage based on user input and/or sensed data) behavior modifications relating to sleep and other suitable characteristics of subject 345. Information module 335 may provide the recommendations via the exemplary techniques described for example above using user interface 320 or any other suitable device or technique.

Also at step 555 when data (e.g., a real-time or near real-time camera data stream of visual sensor 350) is available, information module 335 may utilize artificial intelligence processes to recognize items in an area around subject 345 (e.g., sleep area) and may recommend sleep hygiene protocols based on the recognized items. For example, information module 335 may recommend a revised placement, removal, and/or addition of one or more items. Based on visual data (e.g., data sensed by visual sensor 350), information module 335 may track light levels in an area at or near subject 345, utilize the data in sleep analysis, and make recommendations for actions to take regarding lighting (e.g., adjusting overhead or lamp lighting in a room or other location in which subject 345 sleeps). Based on acoustic data (e.g., data sensed by acoustic sensor 355), information module 335 may track noise in an area at or near subject 345, utilize the data in sleep analysis, and make recommendations for actions to take regarding noise (e.g., suppressing or masking noise from people, devices, animals, outside vehicles, and/or other noise sources that may be heard in a room or other location in which subject 345 sleeps). Based on ambient condition data (e.g., data sensed by ambient condition sensor 380) and/or support data (e.g., data sensed by support sensor 370), information module 335 may track room temperature and humidity and/or bedding or sleep support temperature and humidity in an area at or near subject 345, utilize the data in sleep analysis, and make recommendations for actions to take regarding these factors (e.g., adjusting temperature, humidity, and/or bedding).

Further at step 555, information module 335 may utilize artificial intelligence processes to record and present sleep-related development milestones for subject 345. Results of analysis of information module 335 may be provided to a user as graphs and/or any other suitable visual or audio representation of a sleep cycle of subject 345 (e.g., a child's sleep cycle). Information module 335 may provide feedback congratulating a guardian of subject 345 for providing safe and effective sleep hygiene of subject 345, based on evaluating determined performance against predetermined benchmarks (and/or based on performance criteria that may be determined based on machine learning). Subject to user authorization, system 300 may collect sleep data of subject 345 and transfer the data to outside databases via network 301 to improve learning models in the field of sleep science. When biometric data of a guardian of subject 345 is sensed as described for example above, information module 335 may use such data to map improvements for sleep cycles (e.g., of subject 345 and/or the guardian).

Additionally at step 555, information module 335 may utilize artificial intelligence processes to provide up-to-date sleep science information to users of system 300. Information module may also provide feedback and/or interactively teach guardians of subject 345 about sleep safety and to use good sleep hygiene (e.g., behaviors and actions to take regarding actions leading up to a bed time, napping, eating times and food types as they relate to sleeping times, sleeping conditions such as temperature, noise, and lighting, and/or any other suitable actions) regarding subject 345. Information module 335 may also provide individualized feedback on sleep hygiene for subject 345 that is customized based on performing artificial intelligence operations on the data sensed at step 510.

Further at step 555, information module 335 may utilize artificial intelligence processes to track ongoing patterns (e.g., a plurality of sleep sessions) based on sensing data at step 510 and processing data at step 515 of multiple sleep sessions (e.g., system 300 may sense and process data associated with any desired time span of sleep sessions). System 300 may identify unusual patterns based on the data processed at step 515 and recommend that a guardian of subject 345 consult a medical professional. In addition to providing general (e.g., static) information regarding good sleep hygiene (e.g., age-appropriate sleep behaviors), system 300 may provide individualized recommendation based on artificial intelligence processes performed using the sensed data. System 300 may perform artificial intelligence operations to learn actual guardian and child (e.g., subject 345) behavior of users of system 300 and make recommendations to improve sleep hygiene based on the machine learning. System 300 may also update recommendations based on receiving current scientific research as described for example above. System 300 may provide feedback to users (e.g., a guardian of subject 345) at regular intervals or at any other desired times, and may prompt users to make behavioral changes (e.g., make changes to sleep hygiene practices as described for example herein). System 300 may also track and diagnose medical issues or sleep problems based on an operation of information module 335 as described for example herein. System 300 may also provide research study data (e.g., subject to authorization by users of system 300, system 300 may provide data to external databases via network 301 for use in research studies and other activities to advance the sleep science field). After providing feedback at step 555, system 300 may return to step 510.

At step 560, system 300 (e.g., subsystem 305) may determine whether system 300 should continue monitoring based on the data sensed at step 510 and processed and analyzed at step 515 and/or based on user input (e.g., input by a guardian of subject 345). If continuing monitoring is appropriate, system 300 may return to step 510. If continuing monitoring is not appropriate, system 300 may proceed to step 565, ending process 500 (e.g., ending monitoring of a sleep session).

Any suitable steps of process 500 may be performed simultaneously by system 300. For example, system 300 may perform steps 510, 515, 525, 535, 545, and/or 555 simultaneously. Also for example, system 300 may proceed directly from any of steps 520, 530, 540, and 550 to any other one of steps 520, 530, 540, and 550. For example, system 300 may simultaneously sense data at step 510, process data at step 515, and perform any of steps 525, 535, 545, and/or 555 in any desired order (e.g., or perform any desired steps simultaneously).

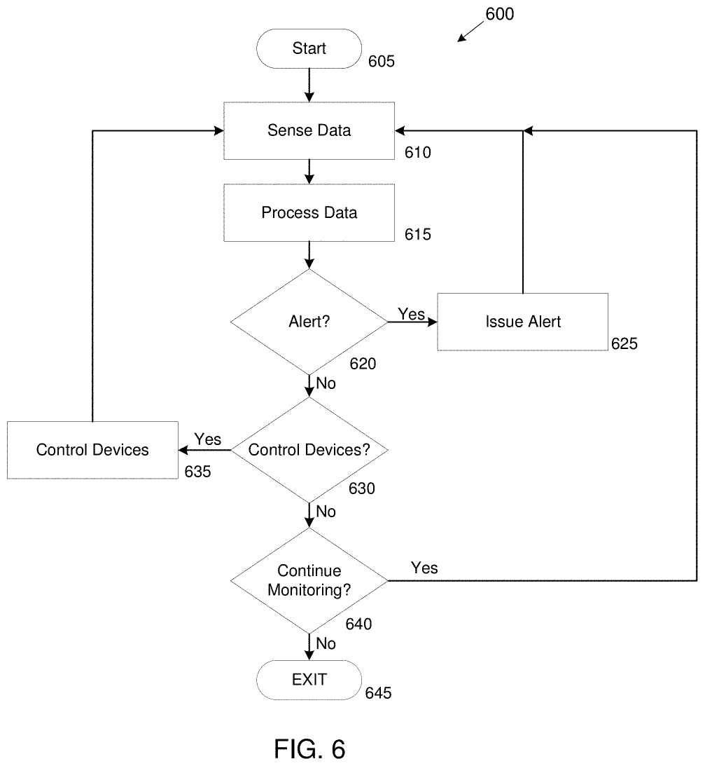

FIG. 6 illustrates another exemplary process of the exemplary disclosed apparatus, system, and method. Process 600 starts at step 605. Steps 610 and 615 may be similar, respectively, to steps 510 and 515. Steps 620 and 625 may be similar, respectively, to steps 520 and 525. Steps 630 and 635 may be similar, respectively, to steps 540 and 545. Steps 640 and 645 may be similar, respectively, to steps 560 and 565.

FIGS. 5 and 6 illustrate different exemplary modes of operation of system 300. For example, process 500 may be a first mode of operation during which guardians of subject 345 are awake. For example, process 500 may be a mode of operation used during daytime naptimes of subject 345 and/or morning or evening times when guardians are awake. Process 600 may be a mode of operation used during late night or early morning periods when guardians of subject 345 are asleep and/or otherwise desire system 300 to operate in a mode consistent with process 600. For example, when guardians of subject 345 are awake (e.g., during an operation of process 500), system 300 may issue alerts at step 525, provide updates at step 535, control devices at step 545, and/or provide feedback at step 555. When guardians of subject 345 are asleep or desire system 300 to operate in a modified mode (e.g., an operation of process 600), system 300 may issue alerts at step 625 and control devices at step 635, but may not provide updates and/or provide feedback (e.g., but may provide such updates and/or feedback based on data sensed and processed during steps 610 and 615 once system 300 has changed its operation back to process 500).

System 300 may switch between a mode of operation of process 500 and a mode of operation of process 600 based on input by a guardian, predetermined time periods during the day, and/or based on data processing by system 300. For example, system 300 may switch between modes of operation (e.g., between process 500 and 600) based on biometric data of a guardian of subject 345 as described for example above. For example, when system 300 determines that a guardian has gone to sleep based on processing sensed biometric data of the guardian, system 300 may switch from process 500 to process 600.

In at least some exemplary embodiments, the exemplary disclosed system may include a sleep pattern module, comprising computer-executable code stored in non-volatile memory, a processor, a sensor array (e.g., sensor array 310) and a device array (e.g., device array 315). The sleep pattern module, the processor, the sensor array, and the device array may be configured to sense data of a sleep subject using the sensor array, process the sensed data, issue an alert to a guardian of the sleep subject based on the processed data, control the device array based on the processed data, and provide sleep pattern modification recommendations to the guardian of the sleep subject based on the processed data. The sensor array may include one or more sensors selected from the group consisting of a camera, a microphone, a biometric sensor, a support sensor, an ambient condition sensor, and a medical sensor. The sensor array may include a video camera, a microphone, a heartrate sensor, and a breathing sensor. The sensor array may include a hygrometer and an ambient temperature sensor. The sensor array may include a medical sensor that senses a biometric attribute of the sleep subject. The device array may include one or more devices selected from the group consisting of a soothing device (e.g., a sound soother and/or a soother using vibration, motion and/or lighting effects), a lighting device, a thermostat, a television, a sound system, a doorbell, and a microwave. The device array may include a soothing device (e.g., a sound soother) that provides sound (e.g., and/or a soothing device that provides soothing lighting, vibration, and/or motion) in a sleep area in which the sleep subject is located, a lighting device that provides light in the sleep area, and a thermostat that controls a temperature in the sleep area and/or in bedding material itself (e.g., a heating or cooling mattress or blanket). The exemplary disclosed system may include a user interface configured to be controlled by the guardian of the sleep subject, wherein the user interface is selected from the group consisting of a smartphone, a smart speaker, and a tablet. The sleep subject may be a child, and the sleep pattern modification recommendations may be recommendations selected from the group consisting of a visual representation of the child's sleep cycle, the child's sleep-related development milestones, and recommended sleep pattern actions for the guardian of the child to take for the child's sleep hygiene. Processing the sensed data may include artificial intelligence operations that produce the sleep pattern modification recommendations. The sleep pattern module, the processor, the sensor array, and the device array may be configured to provide status information including the processed data to the guardian, and prompt the guardian to provide input based on the artificial intelligence operations.

In at least some exemplary embodiments, the exemplary disclosed method may include sensing data of a sleep subject using a sensor array, processing the sensed data, the processed data indicating whether the sleep subject is in a restless state or a deep sleep state, issuing an alert to a guardian of the sleep subject based on the processed data; and controlling a device array based on the processed data, the device array including a soothing device and a lighting device. The exemplary disclosed method may also include providing sleep pattern modification recommendations to the guardian of the sleep subject based on the processed data, activating the soothing device when the sleep subject is in the restless state, and varying a brightness of the lighting device when the sleep subject is in the deep sleep state. For example, a brightness of the lighting device may be increased to provide additional light for a visual device (e.g., camera) to record image data (e.g., higher resolution image data) while the sleep subject is sleeping. The device array may include a thermostat, a television, and a doorbell. The exemplary disclosed method may also include controlling the thermostat to be set at a first temperature when the sleep subject is in the restless state and controlling the thermostat to be at a second temperature that is different from the first temperature when the sleep subject is in the deep sleep state. The exemplary disclosed method may further include reducing an emitting volume of the television and the doorbell when the sleep subject is in the restless state. The exemplary disclosed system may additionally include activating an emergency medical system based on the processed data.

In at least some exemplary embodiments, the exemplary disclosed system may include a sleep pattern module, comprising computer-executable code stored in non-volatile memory, a processor, a sensor array, a device array, and a user interface. The sleep pattern module, the processor, the sensor array, and the device array may be configured to sense data of a sleep subject using the sensor array, process the sensed data, issue an alert to a guardian of the sleep subject based on the processed data, control the device array based on the processed data, and provide sleep pattern modification recommendations via the user interface to the guardian of the sleep subject based on the processed data. The sensor array may include a camera, a microphone, and a breathing sensor. The device array may include a soothing device, a lighting device, and a thermostat. The user interface may be selected from the group consisting of a smartphone, a smart speaker, and a tablet. The user interface may include an artificial intelligence chatbot. Processing the sensed data may include artificial intelligence operations that produce the sleep pattern modification recommendations. The sleep subject may be a child, and the sleep pattern modification recommendations may be recommendations selected from the group consisting of a visual representation of the child's sleep cycle, the child's sleep-related development milestones, and recommended sleep pattern actions for the guardian of the child to take for the child's sleep hygiene.

The exemplary disclosed apparatus, system, and method may provide sleep hygiene and safety information to a guardian of an infant or child that may be individualized to that infant or child. For example, the exemplary disclosed apparatus, system, and method may provide feedback and guidance to a parent or guardian of an infant or young child. The exemplary disclosed apparatus, system, and method may help improve a sleep duration, a timing of sleep, and/or sleep of children in order to improve the lives of both the children and the guardians of the children. The exemplary disclosed apparatus, system, and method may provide a low cost and high availability technique for providing feedback of a child's sleep cycle, which may be used by any user having for example a smartphone or similar computing device.

An illustrative representation of a computing device appropriate for use with embodiments of the system of the present disclosure is shown in FIG. 7. The computing device 100 can generally be comprised of a Central Processing Unit (CPU, 101), optional further processing units including a graphics processing unit (GPU), a Random Access Memory (RAM, 102), a mother board 103, or alternatively/additionally a storage medium (e.g., hard disk drive, solid state drive, flash memory, cloud storage), an operating system (OS, 104), one or more application software 105, a display element 106, and one or more input/output devices/means 107, including one or more communication interfaces (e.g., RS232, Ethernet, Wifi, Bluetooth, USB). Useful examples include, but are not limited to, personal computers, smart phones, laptops, mobile computing devices, tablet PCs, touch boards, and servers. Multiple computing devices can be operably linked to form a computer network in a manner as to distribute and share one or more resources, such as clustered computing devices and server banks/farms.

Various examples of such general-purpose multi-unit computer networks suitable for embodiments of the disclosure, their typical configuration and many standardized communication links are well known to one skilled in the art, as explained in more detail and illustrated by FIG. 8, which is discussed herein-below.

According to an exemplary embodiment of the present disclosure, data may be transferred to the system, stored by the system and/or transferred by the system to users of the system across local area networks (LANs) (e.g., office networks, home networks) or wide area networks (WANs) (e.g., the Internet). In accordance with the previous embodiment, the system may be comprised of numerous servers communicatively connected across one or more LANs and/or WANs. One of ordinary skill in the art would appreciate that there are numerous manners in which the system could be configured and embodiments of the present disclosure are contemplated for use with any configuration.

In general, the system and methods provided herein may be employed by a user of a computing device whether connected to a network or not. Similarly, some steps of the methods provided herein may be performed by components and modules of the system whether connected or not. While such components/modules are offline, and the data they generated will then be transmitted to the relevant other parts of the system once the offline component/module comes again online with the rest of the network (or a relevant part thereof). According to an embodiment of the present disclosure, some of the applications of the present disclosure may not be accessible when not connected to a network, however a user or a module/component of the system itself may be able to compose data offline from the remainder of the system that will be consumed by the system or its other components when the user/offline system component or module is later connected to the system network.

Referring to FIG. 8, a schematic overview of a system in accordance with an embodiment of the present disclosure is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to an exemplary embodiment, as shown in FIG. 8, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present disclosure may or may not utilize one or more routers 202. One of ordinary skill in the art would appreciate that there are numerous ways server 203 may connect to WAN 201 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 203 via WAN 201 or other network in numerous ways. For instance, a component or module may connect to the system i) through a computing device 212 directly connected to the WAN 201, ii) through a computing device 205, 206 connected to the WAN 201 through a routing device 204, iii) through a computing device 208, 209, 210 connected to a wireless access point 207 or iv) through a computing device 211 via a wireless connection (e.g., CDMA, GMS, 3G, 4G) to the WAN 201. One of ordinary skill in the art will appreciate that there are numerous ways that a component or module may connect to server 203 via WAN 201 or other network, and embodiments of the present disclosure are contemplated for use with any method for connecting to server 203 via WAN 201 or other network. Furthermore, server 203 could be comprised of a personal computing device, such as a smartphone, acting as a host for other computing devices to connect to.

The communications means of the system may be any means for communicating data, including image and video, over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

Traditionally, a computer program includes a finite sequence of computational instructions or program instructions. It will be appreciated that a programmable apparatus or computing device can receive such a computer program and, by processing the computational instructions thereof, produce a technical effect.

A programmable apparatus or computing device includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computing device can include any and all suitable combinations of at least one general purpose computer, special-purpose computer, programmable data processing apparatus, processor, processor architecture, and so on. It will be understood that a computing device can include a computer-readable storage medium and that this medium may be internal or external, removable and replaceable, or fixed. It will also be understood that a computing device can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the disclosure as claimed herein could include an optical computer, quantum computer, analog computer, or the like.

Regardless of the type of computer program or computing device involved, a computer program can be loaded onto a computing device to produce a particular machine that can perform any and all of the depicted functions. This particular machine (or networked configuration thereof) provides a technique for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Illustrative examples of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A data store may be comprised of one or more of a database, file storage system, relational data storage system or any other data system or structure configured to store data. The data store may be a relational database, working in conjunction with a relational database management system (RDBMS) for receiving, processing and storing data. A data store may comprise one or more databases for storing information related to the processing of moving information and estimate information as well one or more databases configured for storage and retrieval of moving information and estimate information.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software components or modules, or as components or modules that employ external routines, code, services, and so forth, or any combination of these. All such implementations are within the scope of the present disclosure. In view of the foregoing, it will be appreciated that elements of the block diagrams and flowchart illustrations support combinations of means for performing the specified functions, combinations of steps for performing the specified functions, program instruction technique for performing the specified functions, and so on.

It will be appreciated that computer program instructions may include computer executable code. A variety of languages for expressing computer program instructions are possible, including without limitation C, C++, Java, JavaScript, assembly language, Lisp, HTML, Perl, and so on. Such languages may include assembly languages, hardware description languages, database programming languages, functional programming languages, imperative programming languages, and so on. In some embodiments, computer program instructions can be stored, compiled, or interpreted to run on a computing device, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computing device enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computing device can process these threads based on priority or any other order based on instructions provided in the program code.

Unless explicitly stated or otherwise clear from the context, the verbs “process” and “execute” are used interchangeably to indicate execute, process, interpret, compile, assemble, link, load, any and all combinations of the foregoing, or the like. Therefore, embodiments that process computer program instructions, computer-executable code, or the like can suitably act upon the instructions or code in any and all of the ways just described.

The functions and operations presented herein are not inherently related to any particular computing device or other apparatus. Various general-purpose systems may also be used with programs in accordance with the teachings herein, or it may prove convenient to construct more specialized apparatus to perform the required method steps. The required structure for a variety of these systems will be apparent to those of ordinary skill in the art, along with equivalent variations. In addition, embodiments of the disclosure are not described with reference to any particular programming language. It is appreciated that a variety of programming languages may be used to implement the present teachings as described herein, and any references to specific languages are provided for disclosure of enablement and best mode of embodiments of the disclosure. Embodiments of the disclosure are well suited to a wide variety of computer network systems over numerous topologies. Within this field, the configuration and management of large networks include storage devices and computing devices that are communicatively coupled to dissimilar computing and storage devices over a network, such as the Internet, also referred to as “web” or “world wide web”.

Throughout this disclosure and elsewhere, block diagrams and flowchart illustrations depict methods, apparatuses (e.g., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “component”, “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present disclosure are contemplated for use with any language.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

It will be apparent to those skilled in the art that various modifications and variations can be made to the disclosed apparatus, system, and method. Other embodiments will be apparent to those skilled in the art from consideration of the specification and practice of the disclosed method and apparatus. It is intended that the specification and examples be considered as exemplary only, with a true scope being indicated by the following claims.

What is claimed is:

1. A system, comprising:
a sleep pattern module, comprising computer-executable code stored in non-volatile memory;
a processor;
a sensor array; and
a device array;
wherein the sleep pattern module, the processor, the sensor array, and the device array are configured to:
sense data of a sleep subject using the sensor array;
process the sensed data;
issue an alert to a guardian of the sleep subject based on the processed data;
control the device array based on the processed data; and
provide sleep pattern modification recommendations to the guardian of the sleep subject based on the processed data.

2. The system of claim 1, wherein the sensor array includes one or more sensors selected from the group consisting of a camera, a microphone, a biometric sensor, a support sensor, an ambient condition sensor, and a medical sensor.

3. The system of claim 1, wherein the sensor array includes a video camera, a microphone, a heartrate sensor, and a breathing sensor.

4. The system of claim 1, wherein the sensor array includes a hygrometer and an ambient temperature sensor.

5. The system of claim 1, wherein the sensor array includes a medical sensor that senses a biometric attribute of the sleep subject.

6. The system of claim 1, wherein the device array includes one or more devices selected from the group consisting of a soothing device, a lighting device, a thermostat, a television, a sound system, a doorbell, and a microwave.

7. The system of claim 1, wherein the device array includes a soothing device that provides sound in a sleep area in which the sleep subject is located, a lighting device that provides light in the sleep area, and a thermostat that controls a temperature of the sleep area.

8. The system of claim 1, further comprising a user interface configured to be controlled by the guardian of the sleep subject, wherein the user interface is selected from the group consisting of a smartphone, a smart speaker, and a tablet.

9. The system of claim 1, wherein the sleep subject is a child, and the sleep pattern modification recommendations are recommendations selected from the group consisting of a visual representation of the child's sleep cycle, the child's sleep-related development milestones, and recommended sleep pattern actions for the guardian of the child to take for the child's sleep hygiene.

10. The system of claim 9, wherein processing the sensed data includes artificial intelligence operations that produce the sleep pattern modification recommendations.

11. The system of claim 10, wherein the sleep pattern module, the processor, the sensor array, and the device array are configured to:
provide status information including the processed data to the guardian; and
prompt the guardian to provide input based on the artificial intelligence operations.

12. A method, comprising:
sensing data of a sleep subject using a sensor array;
processing the sensed data, the processed data indicating whether the sleep subject is in a state selected from the group consisting of a restless sleep state, a light sleep state, a deep sleep state, an REM sleep state, and a transitional sleep state;
issuing an alert to a guardian of the sleep subject based on the processed data;
controlling a device array based on the processed data, the device array including a soothing device and a lighting device;
providing sleep pattern modification recommendations to the guardian of the sleep subject based on the processed data;
activating the soothing device when the sleep subject is in the restless sleep state or the light sleep state; and
varying a brightness of the lighting device when the sleep subject is in the deep sleep state or the REM sleep state.

13. The method of claim 12, wherein the device array includes a thermostat, a television, and a doorbell.

14. The method of claim 13, further comprising controlling the thermostat to be set at a first temperature when the sleep subject is in the restless state and controlling the thermostat to be at a second temperature that is different from the first temperature when the sleep subject is in the deep sleep state.

15. The method of claim 13, further comprising reducing an emitting volume of the television and the doorbell when the sleep subject is in the restless sleep state or the light sleep state.

16. The method of claim 12, further comprising activating an emergency medical system based on the processed data.

17. A system, comprising:
a sleep pattern module, comprising computer-executable code stored in non-volatile memory;
a processor;
a sensor array;
a device array; and
a user interface;
wherein the sleep pattern module, the processor, the sensor array, and the device array are configured to:
sense data of a sleep subject using the sensor array;
process the sensed data;
issue an alert via the user interface to a guardian of the sleep subject based on the processed data;
control the device array based on the processed data; and
provide sleep pattern modification recommendations via the user interface to the guardian of the sleep subject based on the processed data;
wherein the sensor array includes a camera, a microphone, and a breathing sensor;
wherein the device array includes a soothing device, a lighting device, and a thermostat; and
wherein the user interface is selected from the group consisting of a smartphone, a smart speaker, and a tablet.

18. The system of claim 17, wherein the user interface includes an artificial intelligence chatbot.

19. The system of claim 17, wherein processing the sensed data includes artificial intelligence operations that produce the sleep pattern modification recommendations.

20. The system of claim 19, wherein the sleep subject is a child, and the sleep pattern modification recommendations are recommendations selected from the group consisting of a visual representation of the child's sleep cycle, the child's sleep-related development milestones, and recommended sleep pattern actions for the guardian of the child to take for the child's sleep hygiene.
